(19) United States
(12) Patent Application Publication
(10) Pub. No.: US 2024/0067828 A1
(43) Pub. Date: Feb. 29, 2024

(54) STABILISATION OF CARBONATE CALCIUM NANOPARTICLES

(71) Applicant: LUXEMBOURG INSTITUTE OF SCIENCE AND TECHNOLOGY (LIST), Esch-sur-Alzette (LU)

(72) Inventors: Lauriane CHUZEVILLE, Esch-sur-Alzette (LU); Jean-Sébastien THOMANN, Esch-sur-Alzette (LU)

(21) Appl. No.: 18/270,477

(22) PCT Filed: Nov. 29, 2021
(86) PCT No.: PCT/EP2021/083361

§ 371 Date: Jun. 29, 2023

(30) Foreign Application Priority Data
Dec. 30, 2020 (LU) LU102356

(51) Int. Cl. C09C 1/02

(52) U.S. Cl. CPC C09C 1/021; C01P 2002/72; C01P 2004/03; C01P 2004/04; C01P 2004/52; C01P 2006/40

(57) ABSTRACT

The disclosure relates to lipid-coated calcium carbonate nanoparticles, said nanoparticles comprising an outer layer and a core being calcium carbonate nanoparticle, wherein said core is vaterite, proto-vaterite, or amorphous calcium carbonate as determined by X-Ray diffraction, wherein said core is at least partially coated with one or more amphiphilic compounds each having one hydrophilic head and at least one hydrophobic tail, remarkable in that the hydrophilic heads are negatively charged and form the outer layer of the nanoparticles, in that the nanoparticles have a surface charge having a ζ-potential below 0 mV as determined by micro-electrophoretic light scattering technology and in that said one or more amphiphilic compounds are PEG-free. The disclosure also relates to methods for forming such nanoparticles, for modulating the electrical charge of such nanoparticles as well as to the uses of such nanoparticles.

### FIELD OF THE DISCLOSURE

The present disclosure relates to lipid-coated calcium carbonate nanoparticles, methods for forming such nanoparticles and uses of such nanoparticles.

### BACKGROUND OF THE DISCLOSURE

Calcium carbonate colloids are of great interest in various industrial fields due to their advantageous properties. Indeed, in the medical field, at acidic pH, they naturally decompose into carbon dioxide and calcium ions, which makes them pH responsive materials for bioimplant, drug delivery purposes etc. In the composite field, they can be used as reinforcing agent or thixotropic agent. In hygiene technology, they can be used as scrubbing materials or teeth cleaning materials.

For all of these applications, nano size colloids are particularly suitable. Indeed, such a size allows the colloids to cross the biological barrier, increase surface area or induce better rheological properties. However, if not stabilized in water, amorphous calcium carbonate (ACC) nanoparticles are not stable which hampers their use especially in biological field.

In the study of Xu C. et al. (Adv. Funct. Mater., 2019, 1808146) entitled “Biodegradable Nanoparticles of Polyacrylic Acid (PAA)—Stabilized Amorphous CaCO₃for Tunable pH-Responsive Drug Delivery and Enhanced Tumor Inhibition”, it is reported as a solution for stabilizing amorphous calcium carbonate nanoparticles in aqueous media, the synthesis of hybrid PAA-ACC NPs encapsulating doxorubicin, obtained by embedding and stabilizing calcium carbonate nuclei into a PAA nanosphere. Such ACC NPs with PAA are hybrid nanocarrier that can release the doxorubicin at a pH ranging between 5.6 and 7.4. It was found that by doping the layers with Sr⁺⁺ or Mg⁺⁺, it was possible to enable drug delivery at a higher pH, namely a pH ranging between 6.0 and 7.7, providing thus acid-resistant nanoparticles. However, the hybrid character of the material and the synthetic nature of PAA does not contribute to its biodegradability within the natural medium to which such nanocarrier is to be used.

In the study of Peng J. Q et al. (J. Controlled Release, 2019, 302, 42-53) entitled “Targeted co-delivery of protein and drug to a tumor in vivo by sophisticated RGD-modified lipid-calcium carbonate nanoparticles,” it is reported calcium carbonate nanoparticles with a bilayer having on its outer surface PEG and a peptide motif responsible for cell adhesion, namely arginyl-glycyl-aspartic acid (i.e. RGD). This self-assembled bilayer conferred protection of the condensed core in serum which mimics in vivo circulating blood. In an acidic environment, the calcium carbonate decomposes, which allows releasing of any drug or protein that could be encapsulated within the nanoparticles. The size dispersion is broad (up to PDI 0.3) which hampers biological applications. The large PDI is probably due to instability (particle coalescence) or process related issue. However, the synthetic route is highly optimized for this specific application and limits the versatility of the nanoparticles. The use of PEGylated lipids, although conferring stealth properties, limits the possible interactions with other molecules and therefore the employability of the particle for other purposes. Furthermore, PEG is now a controverted material due to the anti-PEG induced immunity and biodegradability.

In the studies of Zhao P. et al. (Nanomedicine: Nanotech. Bio. and Med., 2017, 13, 2507-2516; and Acta Biomateriala, 2018, 72, 248-255) entitled “Enhancing anti-tumor efficiency in hepatocellular carcinoma through the autophagy inhibition by miR-375/sorafenib in lipid-coated calcium carbonate nanoparticles” and “MiR-375 delivered by lipid-coated doxorubicin-calcium carbonate nanoparticles overcomes chemoresistance in hepatocellular carcinoma”, the preparation of anionic lipid-coated CaCO₃ core was reported, using a water-in-oil reverse emulsion method. A mixture of cyclohexane and poly-oxyethylene derivative was used, in which a solution of dioleoylphosphatidic acid in chloroform and CaCl₂ was added to form a calcium microemulsion. A carbonate microemulsion comprising among other Na₂CO₃ was prepared and then combined to the calcium microemulsion. Then after centrifugation, calcium carbonate nanoparticles cores were obtained. The cores were then dispersed in chloroform with DSPE-PEG₂₀₀₀, cholesterol and 1,2-dioleoyl-3-trimethylammonium-propane (i.e. DOTAP). The pH-response of these calcium carbonate nanoparticles acting as drug carrier was studied and it was found that they were stable at pH of about 7.4 while at acidic pH (about 5.5-about 6.8), the drug was released. This work suffers from the same draw back as Peng J. Q. et al. (cf. supra) due to the use of PEG and high particle polydispersity. Furthermore, they are employing hazardous organic solvents for the synthesis.

In these previous studies, PEGylated lipids, synthetic polymer matrices or organic solvents were used to stabilize the ACC NPs. Therefore, the method to manufacture these stabilized NPs is not environment-friendly. A solution was suggested in the study of Wang C. et al. (Chem. Comm., 2018, 54, 13080-13083) which is entitled “Facile preparation of phospholipid-amorphous calcium carbonate hybrid nanoparticles: toward controllable burst drug release and enhanced tumor penetration”, it is reported the use of ACC NPs with PEGylated lipids into water-responsive burst drug release in cancer cells. The ACC NPs, encapsulating doxorubicin, were dispersed into ethanol containing a phosphatidylcholine derivative and 1,2-distearoyl-sn-glycero-3-phosphoethanolamine-N-[methoxy(polyethylene glycol)-2000] (i.e. DSPE-PEG₂₀₀₀)

After 24 hours of stirring at 37° C., the mixture was ultrasonicated at 800 W at room temperature for 20 times during 2 seconds and then centrifugated at 3000 rpm during 10 minutes. The obtained supernatant was then injected into water, followed by dialysis to obtain nanoparticles of calcium carbonate encapsulating doxorubicin and protected by a shell of PEGylated phospholipids. In vitro drug release experiments have demonstrated that the doxorubicin is locked within the nanoparticles for about 25 hours at a pH ranging between 5.5 and 7.4. It was also shown that these nanoparticles can enter the cell, in which an endo-lysosome can further degrade the nanoparticles to expose the core of the complex amorphous calcium carbonate/doxorubicin so that the doxorubicin is released due to the aqueous medium and the acid-responsive decomposition of the amorphous calcium carbonate. Although the process is environmental-friendly, it is time consuming and energy consuming (24 h stirring at 37° C.) and PEGylated moieties are still included in the composition of the protective supported lipid bilayer.

Nevertheless, the use of phospholipids bearing several PEG moieties is problematic in the sense that these PEG derivatives are not that much biocompatible neither biodegradable. Thus, the addition of PEGylated lipids in biological membranes composition is known to prevent the calcium-induced fusion of phospholipid membranes, by inhibiting their interaction with Ca⁺⁺ cations (see for example the studies of Magarkar A. et al. (The Journal of Physical Chemistry B, 2012, 116, 4212-4219), entitled “Molecular Dynamics simulation of PEGylated bilayer interacting with salt ions: a model of the liposome surface in the bloodstream”). Furthermore, the use of PEG moieties as a protective layer of a calcium carbonate core renders the nanoparticles sterically hindered, limiting further potential functionalization of such nanoparticles.

A remark on all these studies, is the use of a cationic or negative active ingredients (drug or mi-RNA) also playing a large role in the stabilisation of calcium carbonate nanoparticles by creating a complex. The versatility of such strategies is therefore questionable.

The study of Szcześ A. et al. (J. Therm. Anal. Calorim., 2016, 123, 2357-2365) describes the preparation of liposomes with a membrane made of 1,2-dipalmitoyl-sn-glycero-3-phosphocholine (DPPC) to partially stabilize vaterite. The liposomes were used as a template to precipitate the calcium carbonate and to influence the properties of the obtained calcium carbonate.

There is, therefore, a need to improve the stability, biocompatibility, biodegradability and processability (time of synthesis, use of environmentally-friendly components . . . ) of lipid-coated calcium carbonate nanoparticles, as well as their versatility.

### SUMMARY OF THE DISCLOSURE

According to a first aspect, the disclosure relates to lipid-coated calcium carbonate nanoparticles, said nanoparticles comprising an outer layer and a core being calcium carbonate nanoparticle, wherein said core is one or more selected from vaterite, proto-vaterite, and amorphous calcium carbonate as determined by X-Ray diffraction, wherein said core is at least partially coated with one or more amphiphilic compounds wherein each amphiphilic compound has one hydrophilic head and at least one hydrophobic tail, remarkable in that the hydrophilic heads of said one or more amphiphilic compounds are negatively charged and form the outer layer of the nanoparticles, in that the nanoparticles have a surface charge having a ζ-potential below 0 mV as determined by micro-electrophoretic light scattering technology and in that said one or more amphiphilic compounds are PEG-free.

It was found that the calcium carbonate nanoparticles in which the core is at least partially surrounded with one or more PEG-free amphiphilic compounds, the hydrophilic heads of which being negatively charged and in which the nanoparticles present an outer layer with an overall negative charge, as indicated by the negative values obtained in ζ-potential measurements, are more stable and more versatile than calcium carbonate nanoparticles coated with amphiphilic layers to which hydrophilic groups responsible for steric hindrance have been incorporated. This steric hindrance is restricting the electrostatic interaction between the nanoparticles themselves and other molecules. By contrast, the lipid-coated calcium carbonate nanoparticles of the present disclosure provide a stabilization towards the calcium carbonate that is of the ionic type, since the one or more amphiphilic compounds are PEG-free and subsequently less bulky.

Additionally, such nanoparticles electrostatically stabilized are made through a method that is time-efficient and energy-efficient compared to the methods provided by the state of the art for lipid coated calcium carbonate nanoparticles.

Without being bound by a theory, the fact that the core of the nanoparticles is at least partially coated with amphiphilic layers arranged in a manner that the external surface of the outer layer of the nanoparticles is mostly or totally composed of hydrophilic negatively charged moieties allows having interaction with different organic and/or inorganic compounds, favouring further templating approaches in aqueous media and further functionalization of the nanoparticles. This makes the lipid-coated calcium carbonate nanoparticles of the present disclosure versatile. Furthermore, the absence of bulky chemical moieties, such as PEG, increases the biocompatibility, the biodegradability and the attractiveness of the nanoparticles. The absence of PEGylated additives in the food, cosmetic and/or pharmaceutical industries prevents the emergence of anti-PEG immunity and hypersensitivity/allergic responses and is therefore beneficial in these areas. In fact, in the biomedical field, the apparition of anti-PEG IgG and IgM is responsible for efficacy loss and hypersensitivity reactions.

More specifically, the cosmetic industries are adapting to market trends and to customers who no longer want to buy cosmetics containing PEGylated compounds, and who are looking for increasingly eco-responsible products, hence the need to find new bio-based compounds (and subsequently devoid of synthetic PEG moieties). Formulations containing PEGylated compounds and/or which do not contain enough bio-sourced raw materials are not purchased by a growing percentage of consumers who are nowadays interested in the composition of their products that are eligible to ecological labels, such as COSMOS certification.

The pharmaceutical industries are also looking for substitutes for PEGylated compounds for the stabilization and stealth of nano-vectors, for the immunity reasons mentioned above and for the development of more eco-responsible technologies.

For example, said core is amorphous calcium carbonate as determined by X-Ray diffraction.

Advantageously, said core is totally coated with one or more amphiphilic compounds wherein each amphiphilic compound has one hydrophilic negatively charged head and at least one hydrophobic tail and/or the external surface of the outer layer of the nanoparticles is hydrophilic and/or the external surface of the outer layer of the nanoparticles is negatively charged.

For example, the external surface of the outer layer of the nanoparticles is composed by at least 50% of said negatively charged heads, preferably at least 75%, more preferably at least 90%, even more preferably at least 95%, most preferably at least 99% or the external surface of the outer layer of the nanoparticles is entirely composed of said negatively charged heads. Indeed, the shift of ζ-potential between the unprotected core and the lipid-coated calcium carbonate nanoparticles demonstrate that the majority of the external surface is covered by negatively charged components.

For example, said one or more amphiphilic compounds are arranged into a plurality of bilayers with negatively charged heads, said plurality of bilayers with the negatively charged heads being interconnected between each other. With preference, said plurality of bilayers are infiltrated into the core of the calcium carbonate nanoparticles, the extent of the infiltration ranging between 5% and 40% of the core diameter as determined by CryoTEM analysis, more preferably ranging between 10% and 35%, even more preferably ranging between 15% and 30%.

For example, the outer layer formed by the hydrophilic heads of said one or more amphiphilic compounds has a thickness ranging between 5 nm and 100 nm as determined by CryoTEM analysis, preferably ranging between 10 nm and 90 nm, more preferably ranging between 20 nm and 80 nm, even more preferably ranging between 30 nm and 70 nm.

With preference, the surface charge of the nanoparticles has a ζ-potential below −5 mV as determined by micro-electrophoretic light scattering technology, preferably below −10 mV, more preferably below −15 mV.

With preference, the outer layer of the nanoparticles has a ζ-potential ranging between −75 mV up to below 0 mV as determined by micro-electrophoretic light scattering technology; more preferably between −70 mV and −5 mV, even more preferably between −65 mV and −10 mV, most preferably between −60 mV and −15 mV.

For example, said one or more amphiphilic compounds are lipids selected from lipids with a negatively charged head and with at least one hydrophobic tail; with preference, from lipids selected from phosphatidyl serine (i.e. PS), phosphatidyl glycerol (i.e. PG), phosphatidyl inositol (i.e. PI) and any mixture thereof. These lipids provide a stabilisation to the nanoparticles that is bio-sourced, which is interesting because the current trends in industries, such as food, cosmetic and/or pharmaceutical industries, is to reduce the use of synthetic stabilizers.

For example, said one or more amphiphilic compounds are selected among 2-dioleoyl-sn-glycero-3-phospho-L-serine (i.e. DOPS), 1,2-dihexadecanoyl-sn-glycero-3-phospho-L-serine; 20 1,2-dipalmitoyl-sn-glycero-3-phospho-rac-(1-glycerol); 1,2-diacyl-sn-glycero-3-phospho-1-rac-gylcerol; 1,2-dioleoyl-sn-glycero-3-phospho-1′-myo-inositol; 1,2-dipalmitoyl-sn-glycero-3-phospho-1′-myo-inositol; and any mixture thereof. With preference, said amphiphilic compound is 2-dioleoyl-sn-glycero-3-phospho-L-serine (i.e. DOPS).

For example, said nanoparticles have an average diameter size ranging between 50 nm and 150 nm as determined by scanning electron microscopy, preferably between 80 nm and 130 nm as determined by scanning electron microscopy, more preferably between 70 nm and 100 nm.

For example, the nanoparticles of the first aspect are monodisperse when dispersed in water or aqueous media.

For example, the nanoparticles of the first aspect have a polydispersity index inferior or equal to 0.20 as determined by dynamic light scattering when dispersed in water or aqueous media, preferably inferior or equal to 0.15, more preferably inferior or equal to 0.14, even more preferably inferior or equal to 0.13.

According to a second aspect, the nanoparticles further comprise one or more additional amphiphilic compounds wherein each additional amphiphilic compound has one hydrophilic head and at least one hydrophobic tail, wherein said hydrophilic heads of said one or more additional amphiphilic compounds are positively charged;

wherein the external surface of the outer layer of the nanoparticles comprises, positively charged heads of the one or more additional amphiphilic compounds in addition to the negatively charged heads of the one or more amphiphilic compounds, wherein said positively charged heads being inserted between the negatively charged heads; and/or

wherein at least a part of said one or more additional amphiphilic compounds is arranged into one or more bilayers with positively charged heads, said one or more bilayers with the positively charged heads being interconnected between each other and covering at least partially the outer layer of said nanoparticles. Advantageously, said one or more bilayers with positively charged heads totally cover the outer layer of said nanoparticles. With preference, said one or more bilayers with the positively charged heads have a thickness comprised between 10 nm and 50 nm as determined by dynamic light scattering, more preferably between 20 nm and 40 nm.

For example, said one or more additional amphiphilic compounds are selected from 1,2-dioleoyl-3-trimethylammonium-propane (i.e., DOTAP) and/or 1,2-dioleoyl-sn-glycero-3-ethylphosphocholine. With preference, said additional amphiphilic compound is 1,2-dioleoyl-3-trimethylammonium-propane (i.e., DOTAP).

For example, the outer layer of the nanoparticles further comprising one or more additional amphiphilic compounds has a ζ-potential ranging between −25 mV and +50 mV as determined by micro-electrophoretic light scattering technology, preferably between −10 mV and +45 mV, more preferably between −5 mV and +40 mV, even more preferably between 0 mV and +35 mV.

For example, the ζ-potential of the outer layer of the nanoparticles of the second aspect is greater than the ζ-potential of the outer layer of the nanoparticles of the first aspect.

For example, the nanoparticles of the second aspect are monodisperse when dispersed in water or aqueous media.

For example, the nanoparticles of the second aspect have a polydispersity index inferior or equal to 0.20 as determined by dynamic light scattering when dispersed in water or aqueous media, preferably inferior or equal to 0.15, more preferably inferior or equal to 0.14, even more preferably inferior or equal to 0.13.

According to a third aspect, the disclosure relates to a method for forming lipid-coated calcium carbonate nanoparticles per the first aspect, said method being remarkable in that it comprises the following steps
a) providing calcium carbonate nanoparticles, to form a core selected from one or more of vaterite, proto-vaterite and amorphous calcium carbonate as determined by X-Ray diffraction;
b) dissolving at least one amphiphilic compound in ethanol, to form an ethanolic solution of at least one amphiphilic compound; wherein said one or more amphiphilic compounds are PEG-free;
c) mixing said calcium carbonate nanoparticles provided in step (a) with said ethanolic solution of at least one amphiphilic compound formed in step (b) to form a mixture;
d) injecting said mixture in water to provide a solution of lipid-coated calcium carbonate nanoparticles as defined in the first aspect; and
e) optionally, recovering said lipid-coated calcium carbonate nanoparticles as defined in the first aspect.

With preference, step (d) is performed under stirring.

For example, said one or more amphiphilic compounds are PEG-free and/or are lipids selected from lipids with a negatively charged head and with at least one hydrophobic tail; with preference, from lipids selected from phosphatidyl serine (i.e. PS), phosphatidyl glycerol (i.e. PG), phosphatidyl inositol (i.e. PI) and any mixture thereof.

For example, said one or more amphiphilic compounds are selected among 2-dioleoyl-sn-glycero-3-phospho-L-serine (i.e. DOPS), 1,2-dihexadecanoyl-sn-glycero-3-phospho-L-serine; 1,2-dipalmitoyl-sn-glycero-3-phospho-rac-(1-glycerol); 1,2-diacyl-sn-glycero-3-phospho-1-rac-gylcerol; 1,2-dioleoyl-sn-glycero-3-phospho-1′-myo-inositol; 1,2-dipalmitoyl-sn-glycero-3-phospho-1′-myo-inositol; and any mixture thereof. With preference, said amphiphilic compound is 2-dioleoyl-sn-glycero-3-phospho-L-serine (i.e. DOPS).

For example, the volume ratio between the water and the mixture of calcium carbonate nanoparticles with at least one amphiphilic compounds in ethanol is ranging between 5 and 15, preferably between 7 and 13.

For example, the weight ratio between the at least one amphiphilic compounds and calcium carbonate of the calcium carbonate nanoparticles is ranging between 0.01 and 1, preferably between 0.05 and 0.80, more preferably between 0.10 and 0.70, even more preferably between 0.20 and 0.50.

For example, the step (a) of providing calcium carbonate nanoparticle comprises the following sub-steps:
i. providing calcium chloride;
ii. dissolving said calcium chloride into ethanol to form an ethanolic solution of calcium chloride;
iii. providing a carbonate salt;
iv. placing the ethanolic solution of formed at sub-step (ii) in a first container and the bicarbonate salt provided at sub-step (iii) in at least one second container inside a reaction chamber, preferably a desiccator;
v. decreasing the pressure within said reaction chamber
vi. sealing the reaction chamber under reduced pressure to form an ethanolic suspension of calcium carbonate nanoparticles;
vii. ageing said ethanolic suspension of calcium carbonate nanoparticles at reduced pressure: and
viii. recovering said ethanolic suspension of calcium carbonate nanoparticles.

Advantageously, the reaction chamber does not comprise a desiccant.

With preference, the reaction chamber comprises a diffusion barrier. For example, the concentration of calcium chloride into ethanol is ranging between 0.25 g/L and 8.0 g/L, preferably between 2.0 g/L and 6.0 g/L.

According to a fourth aspect, the disclosure relates to a method for modulating the electrical charge of lipid-coated calcium carbonate nanoparticles as defined per the first aspect, remarkable in that said method for modulating the electrical charge comprises the following steps:
a) providing the lipid-coated calcium carbonate nanoparticles as defined according to the first aspect and/or as produced by the method defined per the third aspect;
b) dissolving said lipid-coated calcium carbonate nanoparticles provided in step (a) in water to form an aqueous solution of lipid-coated calcium carbonate nanoparticles;
c) dispersing at least one additional amphiphilic compound having one hydrophilic head and at least one hydrophobic tail in ethanol to form an ethanolic solution of said at least one additional amphiphilic compound; wherein said hydrophilic head of said at least one additional amphiphilic compound is positively charged;
d) injecting the ethanolic solution of said at least one additional amphiphilic compound of step (c) into the aqueous solution of lipid-coated calcium carbonate nanoparticles of step (b) to form lipid-coated calcium carbonate nanoparticles as defined in the second aspect;
e) optionally, recovering said lipid-coated calcium carbonate nanoparticles as defined in the second aspect.

With preference, step (d) is performed under stirring.

For example, said one or more additional amphiphilic compounds are selected from 1,2-dioleoyl-3-trimethylammonium-propane (i.e., DOTAP) and/or 1,2-dioleoyl-sn-g/ycero-3-ethylphosphocholine. With preference, said additional amphiphilic compound is 1,2-dioleoyl-3-trimethylammonium-propane (i.e., DOTAP).

For example, the weight ratio between the at least one amphiphilic compound and the at least one additional amphiphilic compound is ranging between 0.05 and 2, preferably between 0.07 and 1.5, more preferably between 0.1 and 1.

According to a fifth aspect, the disclosure relates to the use of lipid-coated calcium carbonate nanoparticles as defined in the first aspect and/or in the second aspect as a buffering agent in cancer therapy, or as a drug carrier, or as a contrast agent, or as a template for core-shell nanoparticles, or as a template for hollow nanoparticles.

For example, the disclosure relates to the use of lipid-coated calcium carbonate nanoparticles produced according to the third aspect and/or to the fourth aspect as a buffering agent in cancer therapy, or as a drug carrier, or as a contrast agent, or as a template for core-shell nanoparticles, or as template for hollow nanoparticles.

### DESCRIPTION OF THE FIGURES

FIG. 1: Influence of the presence of at least one desiccant in the reaction chamber.

FIG. 2: Amorphous calcium carbonate nanoparticles growth curves, with and without diffusion barrier.

FIG. 3: Influence of the initial concentration of CaCl₂.

FIG. 4: XRD spectrum of amorphous calcium carbonate nanoparticles.

FIG. 5: SEM image of the amorphous calcium carbonate nanoparticles.

FIG. 6: TEM image of the amorphous calcium carbonate nanoparticles.

FIG. 7: Size distribution analysis by DLS of amorphous calcium carbonate nanoparticles dissolved in ethanol.

FIG. 8: SEM image of the amorphous calcium carbonate nanoparticles in an aqueous medium.

FIG. 9: Size distribution analysis by DLS of amorphous calcium carbonate nanoparticles in water.

FIG. 10: SEM image of the amorphous calcium carbonate nanoparticles stabilized by DOPS.

FIG. 11: CryoTEM image of an amorphous calcium carbonate nanoparticles stabilized by DOPS.

FIG. 12: XRD spectrum of amorphous calcium carbonate nanoparticles stabilized by DOPS.

FIG. 13: Evolution of the size of amorphous calcium carbonate nanoparticles stabilized by DOPS in function of the DOPS/CaCO₃ weight ratio.

FIG. 14: Stability of the size of amorphous calcium carbonate nanoparticles stabilized by DOPS in function of the temperature.

FIG. 15: Size dispersion of amorphous calcium carbonate nanoparticles stabilized by DOPS after 1 day.

FIG. 16: Size dispersion of amorphous calcium carbonate nanoparticles stabilized by DOPS after 85 days.

FIG. 17: ζ-potential measurement of amorphous calcium carbonate (ACC) nanoparticles and amorphous calcium carbonate nanoparticles stabilized by DOPS (ACC/DOPS).

FIG. 18: ζ-potential measurement of amorphous calcium carbonate nanoparticles stabilized by DOPS (ACC/DOPS) and amorphous calcium carbonate nanoparticles stabilized by DOPS after post-functionalization with DOTAP (ACC/DOPS+DOTAP).

FIG. 19: Size distribution of the amorphous calcium carbonate nanoparticles stabilized by DOPS before and after post-functionalization with DOTAP, with a DOPS/DOTAP weight ratio of 0.1.

FIG. 20: Size distribution of the amorphous calcium carbonate nanoparticles stabilized by DOPS before and after post-functionalization with DOTAP, with a DOPS/DOTAP weight ratio of 1.

### DETAILED DESCRIPTION OF THE DISCLOSURE

For the disclosure, the following definitions are given:

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The recitation of numerical ranges by endpoints includes all integer numbers and, where appropriate, fractions subsumed within that range (e.g. 1 to 5 can include 1, 2, 3, 4, 5 when referring to, for example, a number of elements, and can also include 1.5, 2, 2.75 and 3.80, when referring to, for example, measurements). The recitation of endpoints also includes the recited endpoint values themselves (e.g. from 1.0 to 5.0 includes both 1.0 and 5.0). Any numerical range recited herein is intended to include all sub-ranges subsumed therein.

The particular features, structures, characteristics or embodiments may be combined in any suitable manner, as would be apparent to a person skilled in the art from this disclosure, in one or more embodiments.

### Electrostatic Stabilization of Lipid-Coated Calcium Carbonate Nanoparticles

The disclosure concerns lipid-coated calcium carbonate nanoparticles, said nanoparticles comprising an outer layer and a core being calcium carbonate nanoparticle, wherein said core is one or more selected from vaterite, proto-vaterite, and amorphous calcium carbonate as determined by X-Ray diffraction, wherein said core is at least partially coated with one or more amphiphilic compounds each having one hydrophilic head and at least one hydrophobic tail, remarkable in that the hydrophilic heads of said one or more amphiphilic compounds are negatively charged and form the outer layer of the nanoparticles, in that the nanoparticles have a surface charge having a ζ-potential below 0 mV as determined by micro-electrophoretic light scattering technology and in that the one or more amphiphilic compounds are PEG-free.

For example, the external surface of the outer layer of the nanoparticles is composed by at least 60% of said negatively charged heads, preferably at least 75%, more preferably at least 80% or the external surface of the outer layer of the nanoparticles is entirely composed of said hydrophilic heads. Subsequent electro-stabilisation of the nanoparticles follows.

Advantageously, said core is totally coated with said one or more amphiphilic compounds each having one hydrophilic negatively charged head and at least one hydrophobic tail and/or the external surface of the outer layer is hydrophilic. For example, the external surface of the outer layer of the nanoparticles is negatively charged.

The amphiphilic compounds can arrange themselves into a bilayer, which is a polar membrane made of two layers of such amphiphilic compounds in which the one or more hydrophobic tails of the amphiphilic compounds are oriented toward the centre of the bilayer, leaving the hydrophilic heads of the amphiphilic compounds oriented toward the exterior of the bilayer. A hydrophobic protection is thus created in the centre of the bilayer. In membrane biology, fusion is the process by which two initially distinct lipid bilayers merge their hydrophobic core, resulting in one interconnected structure. If this fusion proceeds completely through both leaflets of both bilayers, an aqueous bridge is formed and the internal contents of the two structures can mix. Alternatively, if only one leaflet from each bilayer is involved in the fusion process, the bilayers are said to be hemifused. In hemifusion, the lipid constituents of the outer leaflet of the two bilayers can mix, but the inner leaflets remain distinct. The aqueous contents enclosed by each bilayer also remain separated.

Advantageously, in the nanoparticles of the disclosure, the one or more amphiphilic compounds are arranged into a plurality of bilayers with negatively charged heads, said plurality of bilayers with the negatively charged heads being interconnected between each other. The type of interconnection is rather a hemifusion than a complete fusion. Calcium cations outside the core may ensure the interconnection between the plurality of bilayers with negatively charged heads.

Advantageously, the plurality of bilayers is infiltrated in the core of the calcium carbonate nanoparticle. With preference, said plurality of bilayers is infiltrated into the core of the calcium carbonate nanoparticles, the extent of the infiltration ranging between 5% and 40% of the core diameter as determined by CryoTEM analysis, more preferably ranging between 10% and 35%, even more preferably ranging between 15% and 30%.

Advantageously, said one or more amphiphilic compounds are PEG-free and/or are lipids selected from lipids with a negatively charged head and with at least one hydrophobic tail; with preference, from lipids selected from phosphatidyl serine (i.e. PS), phosphatidyl glycerol (i.e. PG), phosphatidyl inositol (i.e. PI) and any mixture thereof.

For example, said one or more amphiphilic compounds are selected among 2-dioleoyl-sn-glycero-3-phospho-L-serine (i.e. DOPS), 1,2-dihexadecanoyl-sn-glycero-3-phospho-L-serine; 1,2-dipalmitoyl-sn-glycero-3-phospho-rac-(1-glycerol); 1,2-diacyl-sn-glycero-3-phospho-1-rac-gylcerol; 1,2-dioleoyl-sn-glycero-3-phospho-1′-myo-inositol; 1,2-dipalmitoyl-sn-glycero-3-phospho-1′-myo-inositol; and any mixture thereof. With preference, said amphiphilic compound is 2-dioleoyl-sn-glycero-3-phospho-L-serine (i.e. DOPS).

The negative electrical charge on the heads of the one or more amphiphilic compounds allows the one or more amphiphilic compound to interact with Ca⁺⁺ ions through their polar heads as both leaflets of the bilayer are negatively charged. For example, the one or more amphiphilic compounds formed packed layers or packed bilayers. This particular structuration behaviour around the CC NPs creates effective protection against the molecule of water. Indeed, the packing of the lipid multilayers is expected to prevent more effectively the diffusion of water molecules through the lipid layers compared to less packed lipid multilayers.

Crystalline calcium carbonate, under the form of vaterite (μ-CaCO₃) or proto-vaterite, can be used to prepare the CC nanoparticles. Alternatively, and preferably, the core of the calcium carbonate nanoparticles can be amorphous.

To this regard, it is understood that any amorphous calcium carbonate nanoparticles, or vaterite nanoparticles, or proto-vaterite nanoparticles can be used to prepare lipid-coated calcium carbonate nanoparticles as described above. However, to control the size distribution of the lipid-coated nanoparticles, it is preferred to prepare the calcium carbonate nanoparticles. The calcium carbonate nanoparticles can be for example formed via an ethanol-ammonia diffusion method, as follows:
i. providing calcium chloride (such as CaCl₂.6H₂O or CaCl₂.2H₂O);
ii. dissolving said calcium chloride into ethanol to form an ethanolic solution of calcium chloride; preferably using mechanical or magnetic stirring, more preferably using magnetic stirring;
iii. providing a carbonate salt, preferably ammonium carbonate —(NH₄)₂CO₃— or ammonium bicarbonate —NH₄HCO₃—;
iv. placing the ethanolic solution of formed at sub-step (ii) in a first container and the carbonate salt provided at sub-step (iii) in at least one second container inside a reaction chamber, preferably a desiccator;
v. decreasing the pressure within said reaction chamber;
vi. sealing the reaction chamber under reduced pressure to form an ethanolic suspension of calcium carbonate nanoparticles;
vii. ageing said ethanolic suspension of calcium carbonate nanoparticles at reduced pressure: and
viii. recovering said ethanolic suspension of calcium carbonate nanoparticles.

With preference, ethanol is absolute ethanol and/or step (v) is performed with a vacuum pump.

The reaction chamber can be operated in a temperature ranging between 10° C. and 50° C., preferably at room temperature (about 20° C.).

With preference, the first container is covered by a sealing membrane having one or more holes. For example, the sealing membrane is Parafilm punctured with one or more holes.

Advantageously, the concentration of calcium chloride into ethanol is ranging between 0.25 g/L and 8.0 g/L, preferably between 1.0 g/L and 7.0 g/L, more preferably between 2.0 g/L and 6.0 g/L. The ethanolic solution of calcium chloride is placed inside the reaction chamber and within a first container. Within said ranges, it was observed that the calcium carbonate nanoparticles present a narrower size distribution and a smaller mean diameter. When these ranges are considered, the amount of the bicarbonate salt is ranging for example between 0.4 g and 400 g, preferably between 4 g and 40 g. With preference, the total amount of the bicarbonate salt is divided equally and placed into 1 to 10 second containers, different from the first container comprising the ethanolic solution of calcium chloride, preferably into 2 to 8 containers, more preferably into 3 to 6 containers. The one or more second containers have an opening to allow the diffusion of the bicarbonate salt. Advantageously, the size of the opening is ranging between 0.5 cm and 5 cm, preferably between 1 cm and 3 cm. A sealing membrane having small holes can be placed on the one or more second containers, preferably, the second containers are not covered with a sealing membrane having small holes.

For example, the step (v) of decreasing the pressure is the step of reaching a vacuum ranging between 1 kPa and 800 kPa, preferably between 60 kPa and 700 kPa, more preferably between 200 and 500 kPa. For example, the step (vi) of ageing the ethanolic suspension of calcium carbonate nanoparticles at reduced pressure lasts between 5 hours and 80 hours, preferably between 12 hours and 48 hours.

For example, the step (vi) comprises a step of centrifugation of the ethanolic suspension; with preference, the step of centrifugation is performed with a relative centrifugal force ranging between 20,000 g and 45,000 g, preferably between 25,000 g and 40,000 g, and/or for a time ranging between 1 minute and 1 hour, preferably between 5 minutes and 20 minutes. After the step of centrifugation, the obtained supernatant is discarded and the obtained pellet is dispersed in ethanol, preferably in an amount of ethanol ranging between 1 mL and 100 mL. Said dispersion in ethanol is further centrifugated, preferably with a relative centrifugal force ranging between 2,000 g and 10,000 g, more preferably between 3,500 g and 5,000 g; and/or for a time ranging between 1 minute and 1 hour, preferably between 5 and 20 minutes. The pellet is discarded and the supernatant is recovered since it comprises amorphous calcium carbonate nanoparticles in suspension in ethanol. They can be stored in a fridge, for example at 4° C.

The reaction chamber can contain a desiccant. However, advantageously, the reaction chamber does not comprise any desiccant. The desiccant could be placed in the reaction chamber to trap water molecules generated by the decomposition of the bicarbonate salt.

The diffusion area surface (S) corresponding to the surface of the first container opening (i.e. the surface of the calcium chloride ethanolic solution), and z corresponding to the height of that said calcium chloride solution in the first container, with preference, the ratio S/z is ranging between 5 and 30, more preferably between 10 and 20, even more preferably between 12 and 18.

This ratio influences the final calcium carbonate nanoparticles size, as the formation of calcium carbonate nanoparticles relies on the gas (i.e. gas coming from the decomposition of ammonium carbonate or bicarbonate) diffusion from the reaction chamber atmosphere to the solution of calcium chloride. Said gas diffusion is function of the first Fick's law (1), which estimates the gas flux through the gas-liquid interface (J) using the gas diffusion coefficient in the liquid (D), the diffusion area surface (S), the gas concentration (C) and the height of the solution in the first container (z) according to equation (1).

$$J = D \times S \times \frac{\partial C}{\partial z} \qquad ( 1 )$$

Further information about the formation of the calcium carbonate nanoparticles via the ethanol-ammonia diffusion can be found in the studies of Chen S-F. et al. (Chem. Comm., 2013, 49, 9564-9566), entitled “Ethanol assisted synthesis of pure and stable amorphous calcium carbonate nanoparticles” and/or of Som A. et al. (Nanoscale, 2016, 8, 12639-12674). This ethanol-ammonia diffusion method presents the advantage of preventing the crystallization of the amorphous calcium carbonate nanoparticles into crystalline calcium carbonate polymorphs and is achieved without additives.

In an aspect of the present disclosure, a method for forming lipid-coated calcium carbonate nanoparticles is described. Said method comprises the following steps:
a) providing calcium carbonate nanoparticles, to form a core selected from one or more of vaterite, proto-vaterite and amorphous calcium carbonate as determined by X-Ray diffraction;
b) dissolving at least one amphiphilic compound in ethanol, to form an ethanolic solution of at least one amphiphilic compound, wherein said one or more amphiphilic compounds are PEG-free;
c) mixing said calcium carbonate nanoparticles provided in step (a) with said ethanolic solution of at least one amphiphilic compound formed in step (b) to form a mixture;
d) injecting said mixture in water to provide a solution of lipid-coated calcium carbonate nanoparticles; and
e) optionally, recovering said lipid-coated calcium carbonate nanoparticles. With preference, step (d) is performed under stirring.

For example, said one or more amphiphilic compounds are PEG-free and/or are lipids selected from lipids with a negatively charged head and with at least one hydrophobic tail; with preference, from lipids selected from phosphatidyl serine (i.e. PS), phosphatidyl glycerol (i.e. PG), phosphatidyl inositol (i.e. PI) and any mixture thereof.

For example, said one or more amphiphilic compounds are selected among 2-dioleoyl-sn-glycero-3-phospho-L-serine (i.e. DOPS), 1,2-dihexadecanoyl-sn-glycero-3-phospho-L-serine; 1,2-dipalmitoyl-sn-glycero-3-phospho-rac-(1-glycerol); 1,2-diacyl-sn-glycero-3-phospho-1-rac-gylcerol; 1,2-dioleoyl-sn-g/ycero-3-phospho-1′-myo-inositol; 1,2-dipalmitoyl-sn-glycero-3-phospho-1′-myo-inositol; and any mixture thereof. With preference, said amphiphilic compound is 2-dioleoyl-sn-g/ycero-3-phospho-L-serine (i.e. DOPS).

With preference, the calcium carbonate nanoparticles provided in step (a) are prepared via the ethanol-diffusion method exemplified above.

With preference, step (b) of dissolving at least one amphiphilic compound in ethanol is performed by mechanical, ultrasonic or magnetic stirring, more preferably by ultrasonic stirring. When ultrasonic stirring is performed, an ultrasonic bath is used.

Water is preferably ultra-pure water.

It is preferred that the volume ratio between the water and the mixture of calcium carbonate nanoparticles with at least one amphiphilic compound in ethanol is ranging between 5 and 15, preferably between 7 and 13.

The concentration of the one or more amphiphilic compounds in ethanol is advantageously ranging between 0.001 g/L and 0.5 g/L, preferably between 0.01 g/L and 0.4 g/L.

It is preferred that the weight ratio between the at least one amphiphilic compound and calcium carbonate of the calcium carbonate nanoparticles is ranging between 0.01 and 1, preferably between 0.05 and 0.80, more preferably between 0.10 and 0.70, even more preferably between 0.20 and 0.50.

With preference, the step (d) of injecting is performed with a pipette, more preferably with an automatic pipette; and/or under stirring, more preferably under mechanical or magnetic stirring.

For example, the step (d) of injected is performed with strong pipetting consisting of several aspiration cycles, followed by 1 second to 1 min of vortex.

The negatively charged heads of the one or more amphiphilic compounds confer a negative electrical charge on the outer layer of the nanoparticles. Monitoring of ζ-potential before and after stabilisation shows a switch from positive to negative values. The ζ-potential of the outer layer of the nanoparticles is thus below 0 mV as determined by micro-electrophoretic light scattering technology. For example, the ζ-potential is below −5 mV, preferably below −10 mV, more preferably below −15 mV. With preference, the outer layer has a ζ-potential ranging between −75 mV up to below 0 mV as determined by micro electrophoretic light scattering technology; more preferably between −70 mV and −5 mV, even more preferably between −65 mV and −10 mV, most preferably between −60 mV and −15 mV.

For example, said negatively-charged nanoparticles have an average diameter size ranging between 50 nm and 150 nm as determined by scanning electron microscopy, preferably between 70 nm and 130 nm.

The resulting nanoparticles can be stored in the fridge (for example at 4° C.) in water or aqueous media.

When dispersed in water or aqueous media, said nanoparticles are monodisperse and/or said nanoparticles have a polydispersity index inferior to 0.20 as determined by dynamic light scattering, preferably inferior or equal to 0.15, more preferably inferior or equal to 0.14, even more preferably inferior or equal to 0.13.

The presence of the negative electrical charge, combined with the lack of sterically hindered groups at the external surface of the lipid-coated calcium carbonate nanoparticles, allows for further functionalization of the nanoparticles. Indeed, the steric stabilization due to the presence of sterically hindered chemical group such as PEG has been replaced by an electrostatic stabilisation, that renders the nanoparticles versatile. For instance, it is possible to modulate the electrical charge and therefore reduced the negative electrical charge or transform the negative electrical charge into a positive electrical charge.

### Modulation of the electrical charge on the nanoparticles

The modification by post-insertion of the stabilized lipid-coated calcium carbonate nanoparticles described above with at least one additional amphiphilic compound allows modulating the electrical charge of said nanoparticles. In particular, said at least one additional amphiphilic compound has one hydrophilic head and at least one hydrophobic tail, wherein said hydrophilic head is positively charged. It is thus possible to overturn the negative electrical charge of the outer layer of the nanoparticles described above into a positive electrical charge or to reduce the negative electrical charge to provide stabilized lipid-coated calcium carbonate nanoparticles with a less negative electrical charge. For example, said one or more additional amphiphilic compounds are selected from 1,2-dioleoyl-3-trimethylammonium-propane (i.e., DOTAP) and/or 1,2-dioleoyl-sn-g/ycero-3-ethylphosphocholine. With preference, said additional amphiphilic compound is 1,2-dioleoyl-3-trimethylammonium-propane (i.e., DOTAP).

This modification leads in a first embodiment to lipid-coated calcium carbonate nanoparticles in which the external surface of the outer layer of the nanoparticles comprises, in addition to the negatively charged heads of the one or more amphiphilic compounds, positively charged heads of the one or more additional amphiphilic compounds, said positively charged heads being inserted between the negatively charged heads.

In a second embodiment, this modification leads to lipid-coated calcium carbonate nanoparticles in which at least a part of the one or more additional amphiphilic compounds is arranged into one or more bilayers with positively charged heads, said one or more bilayers with the positively charged heads being interconnected between each other and covering at least partially the outer layer of said nanoparticles. The type of interconnection is again rather an hemifusion than a complete fusion. With preference, the one or more bilayers with positively charged heads have a thickness comprised between 10 nm and 50 nm as determined by dynamic light scattering, preferably between 20 nm and 40 nm. Advantageously, all the one or more additional amphiphilic compounds are arranged into one or more bilayers with positively charged heads, said one or more bilayers with the positively charged heads being interconnected between each other and covering at least partially the outer layer of said nanoparticles.

In a third embodiment, this modification leads to lipid-coated calcium carbonate nanoparticles in which the external surface of the outer layer of the nanoparticles comprises, in addition to the negatively charged heads of the one or more amphiphilic compounds, positively charged heads of the one or more additional amphiphilic compounds, said positively charged heads being inserted between the negatively charged heads and wherein at least a part of said one or more amphiphilic compounds is arranged into one or more bilayers with positively charged heads, said one or more bilayers with the positively charged heads being interconnected between each other and covering at least partially the outer layer of said nanoparticles. The structure of the modulated lipid-coated calcium carbonate nanoparticles often corresponds to such mix between insertion of the positively charged heads between the negatively charged heads and the formation of one or more bilayers with positively charged heads covering at least partially the outer layer of the nanoparticles.

In all three embodiments, the outer layer of the nanoparticles further comprising one or more additional amphiphilic compounds has a ζ-potential ranging between −25 mV up to +50 mV as determined by micro-electrophoretic light scattering technology, preferably between −10 mV and +45 mV, more preferably between −5 mV and +40 mV, even more preferably between 0 mV and +35 mV. Also, in all three embodiments, the ζ-potential of the outer layer of the nanoparticles is greater than the ζ-potential of the outer layer of the nanoparticles devoid of one or more additional amphiphilic compounds.

Therefore, it is provided for a method for modulating the electrical charge of lipid-coated calcium carbonate nanoparticles that have been stabilized. Said method for modulating the electrical charge comprises the following steps:
a) providing electro-stabilized lipid-coated calcium carbonate nanoparticles;
b) dissolving the electro-stabilized lipid-coated calcium carbonate nanoparticles provided in step (a) in water to form an aqueous solution of electro-stabilized lipid-coated calcium carbonate nanoparticles;
c) dispersing at least one additional amphiphilic compound having one hydrophilic head and at least one hydrophobic tail in ethanol to form an ethanolic solution of said at least one additional amphiphilic compound; wherein said hydrophilic head of said at least one additional amphiphilic compound is positively charged;
d) injecting the ethanolic solution of the additional amphiphilic compound of step (c) into the aqueous solution of electro-stabilized lipid-coated calcium carbonate nanoparticles of step (b) to form lipid-coated calcium carbonate nanoparticles in which the electrical charge has been modulated.

With preference, step (d) is performed under stirring.

For example, said one or more additional amphiphilic compounds are selected from 1,2-dioleoyl-3-trimethylammonium-propane (i.e., DOTAP) and/or 1,2-dioleoyl-sn-glycero-3-ethylphosphocholine. With preference, said additional amphiphilic compound is 1,2-dioleoyl-3-trimethylammonium-propane (i.e., DOTAP).

Optionally, a step (e) of recovering the lipid-coated calcium carbonate nanoparticles in which the electrical charge has been modulated is performed after step (d).

With preference, the concentration of the one additional amphiphilic compound in ethanol is ranging between 0.01 g/L and 1 g/L, preferably between 0.1 g/L and 0.5 g/L.

With preference, step (g) of dissolving at least one additional amphiphilic compound in ethanol is performed by mechanical, ultrasonic or magnetic stirring, more preferably by ultrasonic stirring. When ultrasonic stirring is performed, an ultrasonic bath is used.

It is preferred that the weight ratio between the at least one amphiphilic compound and the at least one additional amphiphilic compound is ranging between 0.05 and 2, preferably between 0.7 and 1.5, more preferably between 0.5 and 1.2, even more preferably between 0.1 and 1.

With preference, the step (d) of injecting is performed with a pipette, more preferably with an automatic pipette; and/or under stirring, more preferably under mechanical or magnetic stirring. For example, the step (d) of injecting is performed by strong pipetting (i.e. several fast aspiration cycles) followed by vortex during 1 second to 1 minute.

Whatever the electrical charge of the lipid-coated calcium carbonate nanoparticles of the present disclosure, the nanoparticles are monodisperse and/or show a polydispersity index inferior or equal to 0.20 as determined by dynamic light scattering, preferably inferior or equal to 0.15, more preferably inferior or equal to 0.14, even more preferably inferior or equal to 0.13. Even after modification by post-insertion in the electro-stabilized lipid-coated calcium carbonate nanoparticles of at least one additional amphiphilic compound, when dispersed in water or aqueous media, said nanoparticles are monodisperse and/or said nanoparticles have a polydispersity index inferior or equal to 0.20 as determined by dynamic light scattering, preferably inferior or equal to 0.15, more preferably inferior or equal to 0.14, even more preferably inferior or equal to 0.13.

### Use of Lipid-Coated Calcium Carbonate Nanoparticles

The nanoparticles of the present disclosure can be used as a buffering agent, or as a drug carrier, or as a contrast agent or as a template for core-shell nanoparticles or as a template for hollow nanoparticles.

Buffering Agent:

The dissolution of lipid-coated calcium carbonate nanoparticles in vivo at tumor site increases pH asymptotically to 7.4 as described in the work of A. Som et al., entitled “Monodispersed calcium carbonate nanoparticles modulate local pH and inhibit tumor growth in vivo” (Nanoscale, 2016, 8, 12639-12647). This study shows that calcium carbonate nanoparticles at slightly acidic pH conditions (6.2), like the pH conditions of some tumours microenvironment, dissolve and increase the pH progressively to 7.4, resulting in an inhibition of the tumour growth. According to the study, a size range around 100 nm would be optimal for this application.

Calcium carbonate nanoparticles dissolve in acidic pH, from 0 to 6.8, with different kinetics according the pH. The lipid-coated calcium carbonate nanoparticles of the present disclosure share these properties.

Drug Carrier:

Hydrophobic or hydrophilic drugs can be carried with the lipid-coated calcium carbonate nanoparticles. An example of a hydrophilic drug is doxorubicin, as shown in the paper of Wang C., et al. (cf. supra). The lipid-coated calcium carbonate nanoparticles of the present disclosure are suitable for these applications.

Contrast Agent:

The lipid-coated calcium carbonate nanoparticles can be used as a contrast agent, preferably as an ultrasound contrast agent. This has potential application in medical imaging, notably in tumor detection. Indeed, as it is described in the study of Min K. H. et al. (ACS Nano, 2015, 9, 134-145) entitled “pH-Controlled Gas-Generating Mineralized Nanoparticles: A Theranostic Agent for Ultrasound Imaging and Therapy of Cancers”, the release of carbon dioxide is an intrinsic property of amorphous calcium carbonate nanoparticles. The generation of CO₂ happens during the dissolution of calcium carbonate in acidic conditions (pH 0 to 6.8). Therefore, the generation of a bubble of CO₂, potentially in combination with the action of dyes encapsulated within the core of the nanoparticles, allows the use of the lipid-coated calcium carbonate nanoparticles of the disclosure as a contrast agent. The potential encapsulated dyes can be one or more of calcein, rhodamine, methylene blue and indocyanine green, preferably indocyanine green.

### Test and Determination Methods

### X-Ray Diffraction (XRD)

The XRD measurements were performed on an XRD X'Pert Pro in relection mode with a Cu K_alpha tube mounted (45 kV, 40 mA). The primary optics contained a programmable divergence slit whereas the secondary optics consisted of a PIXcel detector run in 1D mode with a programmable antiscatter slit. Before measuring the sample, the nanoparticles containing solution was pipeted on a zero background holder and covered with a thin Kapton film in order to prevent the solvent from evaporating. During the measurement, the zero background holder was spun.

### CryoTEM

The purpose of the CRYO-TEM analysis is to determine the arrangement of the amphiphilic bilayers surrounding calcium carbonate core. The samples were frozen with liquid nitrogen in carbon grids by FEI tool™ for sample preparation. Analyses were performed using FEI Titan Krios™ CRYO-TEM operated at 200 kV.

### Nano Tracking Analysis (NTA)

Nanoparticles Tracking Analysis (NTA) is a light scattering method which relates the rate of Brownian motion to particle size. This method allows direct and real time visualizing and analysing of the NPs in liquids. During NTA measurement, NPs are illuminated by a focused laser beam and analysed by the light scattered by each individual particle in the microscope onto the image sensor of a charge-coupled device (CCD) camera. This measurement uses the temperature and the viscosity of the liquid to calculate particle size through the Stokes-Einstein equation.

The Nanosight® NS500 was used to characterize sample size dispersion and nanoparticle concentration. The camera used is the sCMOS, with the laser Blue405. The temperature control was set at 25° C. for all analysis. Samples were diluted 1/1000 prior to analysis in ultrapure water.

### Scanning Electron Microscopy (SEM)

SEM images were obtained using Focus Ion Beam (FIB) scanning electronic microscope (model: Helios Nanolab 650), operating at a voltage of 2-30 Kv and current of 13 to 100 pA. The samples are prepared as followed: a drop of solution is deposited on copper tape and left to dry. The samples must be analysed right after drying, to avoid contamination by air moisture. Ideally, the calcium carbonate core dispersed in EtOH can be prepared in a room with a controlled air moisture. Measurements were done in both feel free mode and immersion mode. SEM images were analysed using ImageJ software.

### Dynamic Light Scattering (DLS)

The mean size and the polydispersity index PDI) of the nanoparticles were measured using a Malvern Zetasizer Nano-ZS90 instrument (UK). Samples were not diluted before analysis. Refractive index of polystyrene (1.58654 at 632.8 nm) was used as an internal standard value. Measurements were done with a disposable PS (polystyrene) microcuvette at 25° C. with 120 seconds equilibration time. The dispersants water or EtOH were used according to the sample dispersant. To confirm the reproducibility, three measurements were carried out in each sample. A new cuvette was used for each sample. DLS measures the hydrodynamic diameter of the particles in the given solvent and the conditions of measurement. Therefore, the size of obtained by DLS is usually bigger than the size obtained by using the scanning electron microscope, which measures the actual size of the nanoparticles after drying.

### Micro-electrophoretic light scattering technology

Micro-electrophoretic light scattering technology allows to determine the ζ-potential. The ζ-potential of a nanoparticles in suspension or in solution represents the electric charge coming from ions surrounding the particles in solution. The ζ-potential represents the intensity of the electrostatic or electrical repulsion/attraction between particles. It is one of the fundamental parameters known to affect stability. The ζ-potential properties were investigated in deionized water for lipid-coated calcium carbonate nanoparticles) and in EtOH for amorphous calcium carbonate nanoparticles. These measurements have been made without performing any filtration nor size exclusion nor dilution prior to analysis. ζ-potential has been measured by using Malvern Nano Zetasizer®. In deionized water, at a pH value of 6-7, in EtOH no pH value could be measured.

### EXAMPLES

The embodiments of the present disclosure will be better understood by looking at the example below.

### Amorphous Calcium Carbonate Nanoparticles

In this example, amorphous calcium carbonate nanoparticles are formed as follows:

A solution of calcium chloride hexahydrate in ethanol is prepared. The concentration of calcium chloride hexahydrate is 4.4 g/mL. Magnetic stirring has been used to achieve the dissolving. This solution has been placed in a first container within desiccator operated at room temperature (25° C.). The first container is covered with a sealing membrane of Parafilm punctured with several small holes. The ratio S/z has been determined to be equal to 15. In the desiccator, 4 second containers with an opening of 2 cm and comprising each 10 g of solid ammonium bicarbonate have been placed around the first container. The pressure within the desiccator was decreased, reaching 20 kPa. Once this pressure reached, the desiccator is sealed and the ethanolic suspension of calcium carbonate nanoparticles is then aged for 24 hours under reduced pressure. After centrifugation, performed at 41,000 g during 10 minutes, the supernatant is discarded and the pellet is dispersed in 10 mL of ethanol. The pellet is further centrifugated at 4,000 g for 10 minutes and the supernatant is this time recovered. The amorphous calcium carbonate nanoparticles (AAC NPs) in suspension in ethanol are then stored in a fridge at 4° C.

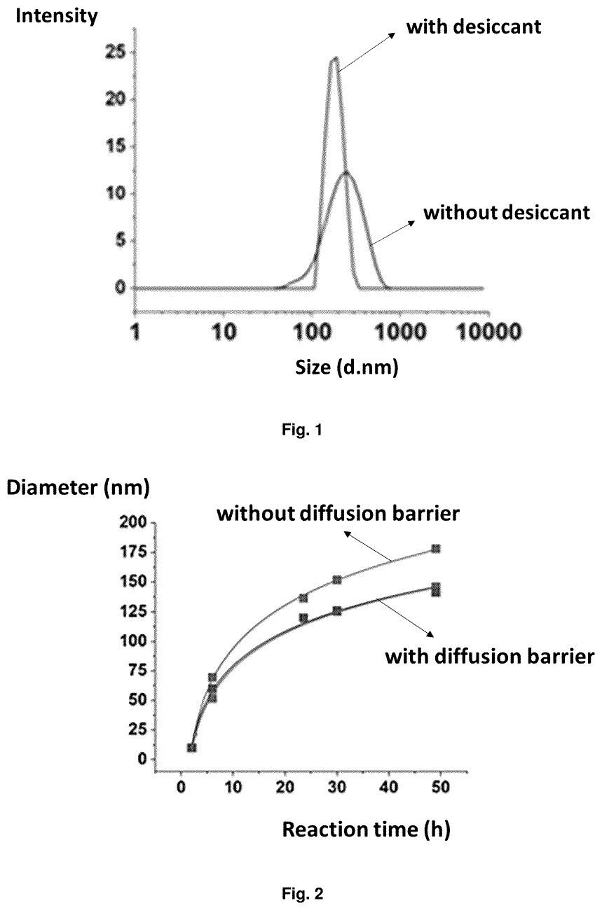

It was observed that the absence of a desiccant allows increasing the concentration of the calcium carbonate nanoparticles and narrowing the size distribution of the nanoparticles, as shown in FIG. 1. The size distribution measured by DLS is narrower when the desiccant is removed. Also, we visually observed a more transparent solution when the desiccant is present in the chamber, evidencing a decrease of the particle concentration when the desiccant is present in the chamber. Removing the desiccant allowed to increase the concentration of nanoparticles and obtain better control of the size

FIG. 2 shows that the incorporation of a diffusion barrier to the first container containing calcium chloride solution in EtOH leads to the obtention of calcium carbonate nanoparticles with smaller size by comparison with the case where a diffusion barrier is not set up. Visual observation and weight concentration comparison after centrifugation indicate a lower concentration by comparison with the case where a diffusion barrier is not set up. No significant change in shape and crystallinity was evidenced.

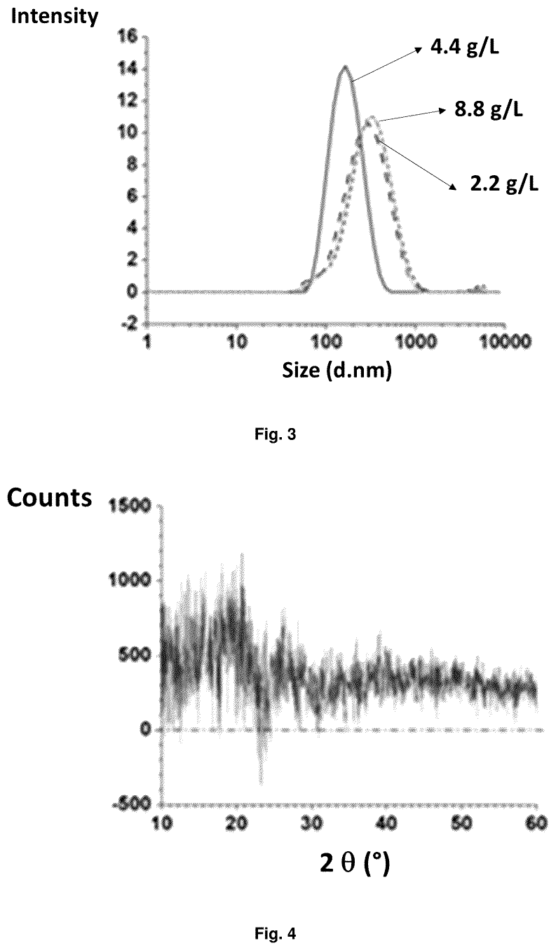

FIG. 3 shows that at a concentration of 4.4 g/L, narrower size distribution and a smaller mean diameter is obtained compared to other concentrations (2.2 g/L or 8.8 g/L).

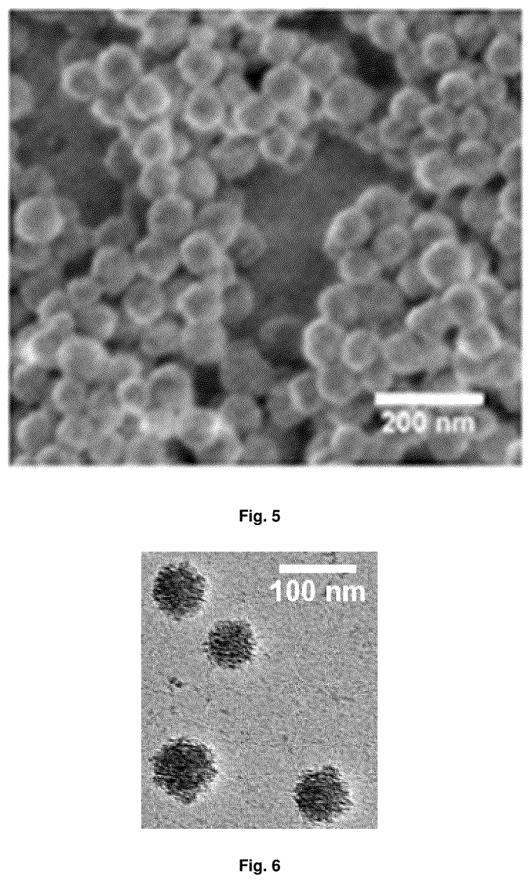

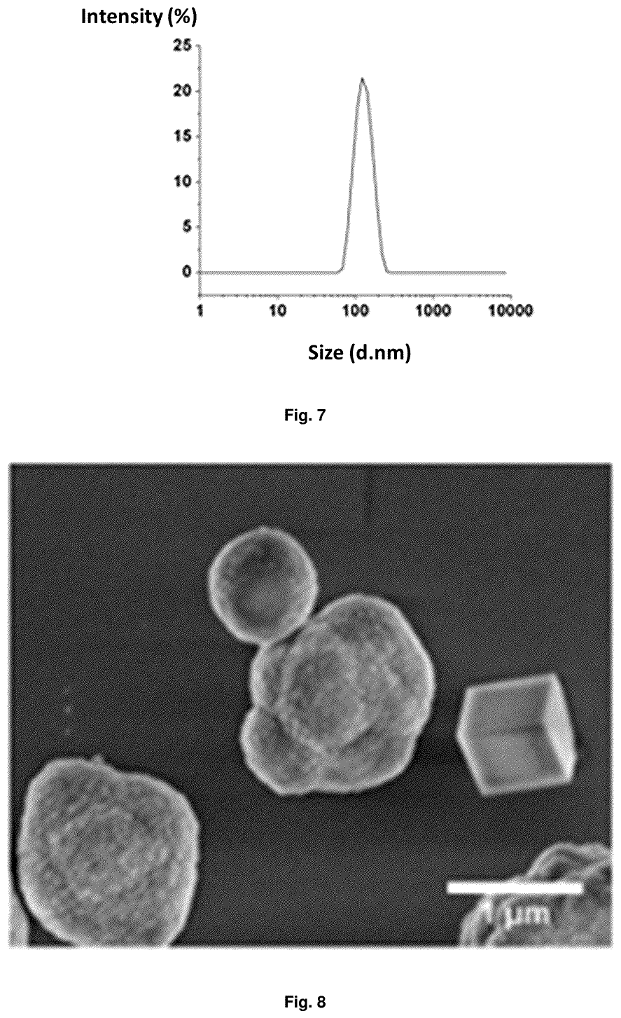

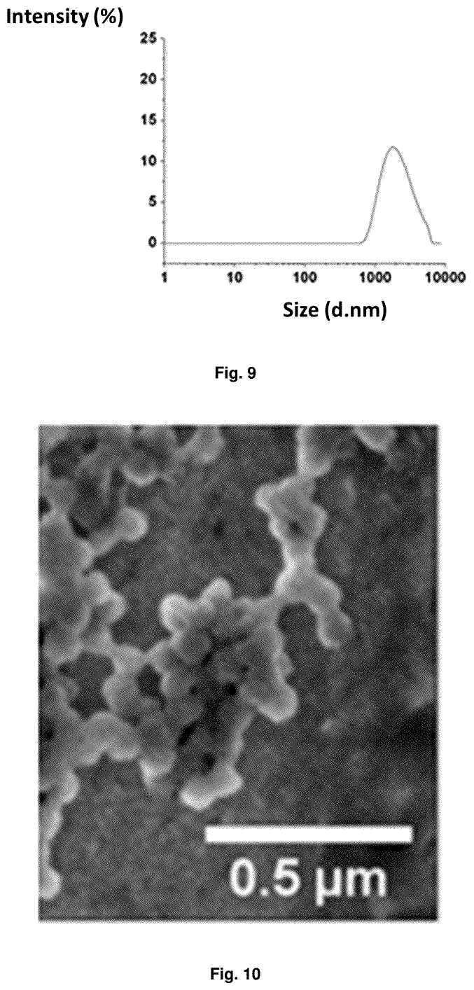

FIG. 4 is an XRD spectrum of the calcium carbonate nanoparticles, evidencing that the calcium carbonate is amorphous. FIG. 5 is an SEM image, showing the non-crystalline spherical calcium carbonate nanoparticles with an average diameter of 70 nm. FIG. 6 is a TEM image, showing the non-crystalline spherical calcium carbonate nanoparticles. FIG. 7, showing the size distribution analysis by DLS confirms that the nanoparticles are monodisperse, with a polydispersity index below 0.05. The size dispersion in DLS is centred on 127 nm, which is a higher value compared to the SEM measurement. This is due to the hydrodynamic diameter of the amorphous calcium carbonate nanoparticles dissolved in ethanol. The shape (FIG. 8) and the size (FIG. 9) of the nanoparticles have been determined in presence of water, revealing the instability of the amorphous calcium carbonate nanoparticles in an aqueous medium. FIG. 8 is an SEM image which confirms the presence of large microparticles with crystalline polymorphs identifiable from their characteristic shape. Those microparticles are vaterite aggregates and trigonal rhombohedral calcite. FIG. 9 indicates that the polydispersity index of the amorphous calcium carbonate particles in an aqueous medium is above 0.25 and the size dispersion is now centred on the micrometre scale.

### Lipid-Coated Amorphous Calcium Carbonate Nanoparticles

To stabilize the amorphous calcium carbonate nanoparticles, the core of amorphous calcium carbonate has been coated with one or more amphiphilic compounds, said one or more amphiphilic compounds having one hydrophilic head at least one hydrophobic tail, wherein the hydrophilic head is negatively charged.

A solution of DOPS in ethanol at a concentration of 0.25 g/L has been prepared. DOPS is dissolved in ethanol by ultrasonic stirring, using an ultrasonic bath with 37 kHz intensity and 100% power. Amorphous calcium carbonate nanoparticles in ethanol are provided and mixed with the ethanolic solution of DOPS, at a weight ratio DOPS/CaCO₃ of 0.3. The mixture is then injected with an automatic pipette with strong pipetting into ultrapure water, at a volume ratio water/mixture of 9 under stirring. The injection is followed by 15 seconds vortex. The strong pipetting comprises several aspiration cycles with the automatic pipette. The resulting aqueous solution of DOPS-coated amorphous calcium carbonate nanoparticles is then stored in the fridge at 4° C.

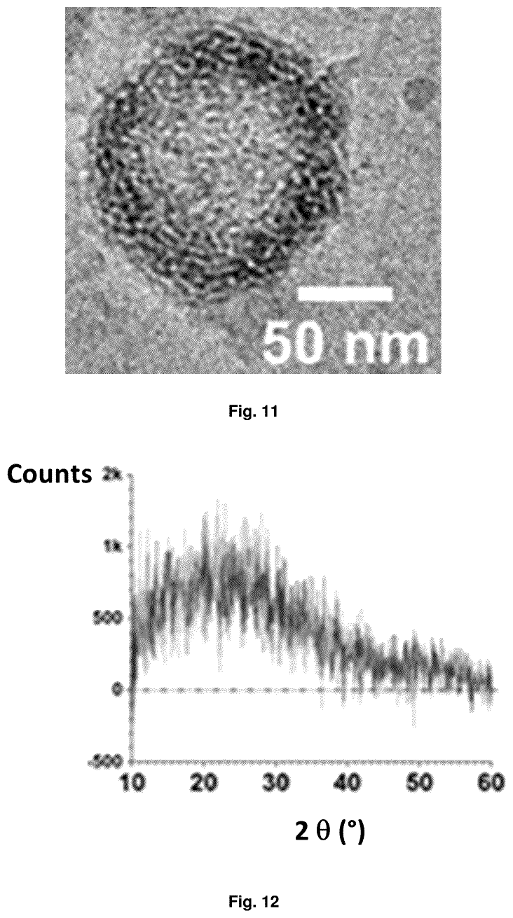

When compared to the study of Wang C. et al. (see supra), the contact time between the lipids and amorphous calcium carbonate nanoparticles is reduced from 24 hours to 1 or 2 minutes. In the study of Wang C. et al., the phospholipids seem to structure themselves around ACC NPs, forming a supported lipid bilayer. The aim of using non PEGylated lipids with a negatively charged polar head and at least one hydrophobic tail was to preserve an effective structuration of the lipids around ACC NPs and obtain an effective stabilisation in water or aqueous media with a reduced contact time before injection. Unexpectedly, as shown in FIG. 11, the structuration obtained with lipids having a negatively charged polar head and at least one hydrophobic tail is completely different, with infiltration of lipid multilayers in the amorphous calcium carbonate core. As shown in Table 1, several phospholipids with different chain length, polar head and number of unsaturations were tested as well as a cationic surfactant (DOTAP) to evaluate the importance of the negatively charged polar heads group in the effectiveness of the stabilisation of amorphous calcium carbonate nanoparticles in water or aqueous media.

TABLE 1
Comparison between different amphiphilic compounds stabilizing
the core of amorphous calcium carbonate nanoparticles

| | Size | | Concentration** |
|---|---|---|---|
| Amphiphilic compounds | (nm)* | PDI* | (particles/ml) |
| 16:0 PC^{a} | 326 | 0.322 | 1.25*10¹¹ |
| DPPC/DSPE-PEG₂₀₀₀^{b} | N/A | N/A | N/A |
| (20/80 wt./wt.) | | | |
| 16:0 PG^{c} | 142.7 | 0.017 | 2.07*10¹¹ |
| EPG^{d} (C12-C24 + C12-C24 | 148.7 | 0.022 | 1.88*10¹¹ |
| unsaturated) | | | |
| 18:1 PS^{e} | 162.5 | 0.18 | 1.46*10¹¹ |
| 18:1 TAP^{f} | 71 | 0.307 | N/A |

*as determined by DLS
**as determined by NTA
N/A non-applicable
^{a}16:0 PC stands for 1,2-dipalmitoyl-sn-glycero-3-phosphocholine
^{b}DSPE-PEG₂₀₀₀ stands for 1,2-distearoyl-sn-glycero-3-phosphoethanolamine-N-[methoxy(polyethylene glycol)-2000]
^{c}16:0 PG stands for phosphatidylglycerol
^{d}EPG stands for egg phosphatidylglycerol
^{e}18:1 PS stands for 2-dioleoyl-sn-glycero-3-phospho-L-serine
^{f}18:1 TAP stands for 1,2-dioleoyl-3-trimethylammonium-propane

An effective stabilisation with narrow size distributions is observed for the phospholipids PG and DOPS. Table 1 shows an absence of stabilization for DPPC, DPPC:DSPE-PEG₂₀₀₀ and DOTAP. The head group charge appears therefore to be determinant to provide efficient stabilisation of amorphous calcium carbonate nanoparticles in water with reduced contact time and non-PEGylated phospholipids.

Surprisingly, using PC (i.e phosphatidylcholine) head group is not providing an effective stabilisation of the amorphous calcium carbonate nanoparticles in water even with the addition of PEGylated lipids, with a limited contact time between amorphous calcium carbonate nanoparticles and the lipid mix before injection in water. A mix of DPPC:DSPE-PEG₂₀₀₀ was tested with a weight ratio DPPC:DSPE-PEG₂₀₀₀ of 20/80 according to the study of Wang C. et al., to evaluate the impact of the reduction of the contact time between the amorphous calcium carbonate nanoparticles and the phospholipids. The use of DPPC:DSPE-PEG₂₀₀₀ in a protective layer for amorphous calcium carbonate nanoparticles was found ineffective with a contact time of 2 minutes. Bubbling was observed during the ethanol injection process. This is the consequence of the dissolution of the amorphous calcium carbonate nanoparticles during the process.

The addition of PEG is not enough to provide effective stabilisation of amorphous calcium carbonate nanoparticles in the absence of non-PEGylated lipids with a negatively charged polar head group and at least one hydrophobic tail. An enhanced contact time between the nanoparticles and the lipid mix is required at 37° C. when zwitterionic polar head lipids are used in combination of PEG for the stabilisation of calcium carbonate nanoparticles in water according to literature.

To reduce the contact time, one or more amphiphilic compounds, in particular non-PEGylated lipids, with a negatively charged polar head group and at least one hydrophobic tail are required.

The stabilisation is efficient with PG and with DOPS phospholipids. As these phospholipids were tested with different chain length and/or unsaturation degree, the stabilisation efficiency does not depend on these factors.

TABLE 2
comparison of the stabilisation efficiency with amphiphilic molecules
with different chain lengths and unsaturation number.

| Amphiphilic | | Concentration* |
|---|---|---|
| compound | Mean diameter (nm) | (particles/mL) |
| 12:0 PS | 97.2 +/− 2.6 | 1.20*10¹¹ |
| 14:0 PS | 100.4 +/− 3.9 | 1.79*10¹¹ |
| 16:0 PS | 97.4 +/− 1.6 | 2.31*10¹¹ |
| 18:0 PS | 112.1 +/− 6.2 | 1.16*10¹¹ |
| 18:1 PS | 97.0 +/− 1.4 | 1.46*10¹¹ |
| 16:0 PG | 104.1 +/− 1.4 | 2.07*10¹¹ |
| EPG | 120.2 +/− 4.6 | 1.88*10¹¹ |

*as determined by NTA
^{a} 12:0 PS stands for 1,2-dilauroyl-sn-glycero-3-phospho-L-serine
^{b} 14:0 PS stands for 1,2-dimyristoyl-sn-glycero-3-phospho-L-serine
^{c} 16:0 PS stands for 1,2-dipalmitoyl-sn-glycero-3-phospho-L-serine
^{d} 18:0 PS stands for 1,2-distearoyl-sn-glycero-3-phospho-L-serine
^{e} 18:1 PS stands for 1,2-dioleoyl-sn-glycero-3-phospho-L-serine
^{f} 16:0 PG stands for phosphatidylglycerol
^{g} EPG stands for egg phosphatidylglycerol

FIG. 10 shows the SEM image of amorphous calcium carbonate nanoparticles stabilized by DOPS. It reveals monodisperse nanoparticles with an average size of 90 nm. By comparison with the FIG. 5, in which the average size of the non-coated amorphous calcium carbonate nanoparticles was determined to be of 70 nm, there is a size increase which testifies from the structuration of a layer of one or more amphiphilic compounds around the core of amorphous calcium carbonate. The multilayers of DOPS stabilizing the core of amorphous calcium carbonate nanoparticles can be seen in FIG. 11, which is a CryoTEM image of the stabilized nanoparticles. Indeed, it was not possible to image the multilayers on DOPS by classic SEM.

FIG. 11 reveals that the structuration does not lead to a supported lipid bilayer around the core of amorphous calcium carbonate. Instead, several layers are observed, interconnected and/or hemifused to each other and infiltrated in the calcium carbonate core, creating thus effective lipid protection around the nanoparticles against the molecules of water. X-Ray Diffraction studies, as shown in FIG. 12, have proven that the amorphous structure of the calcium carbonate is kept.

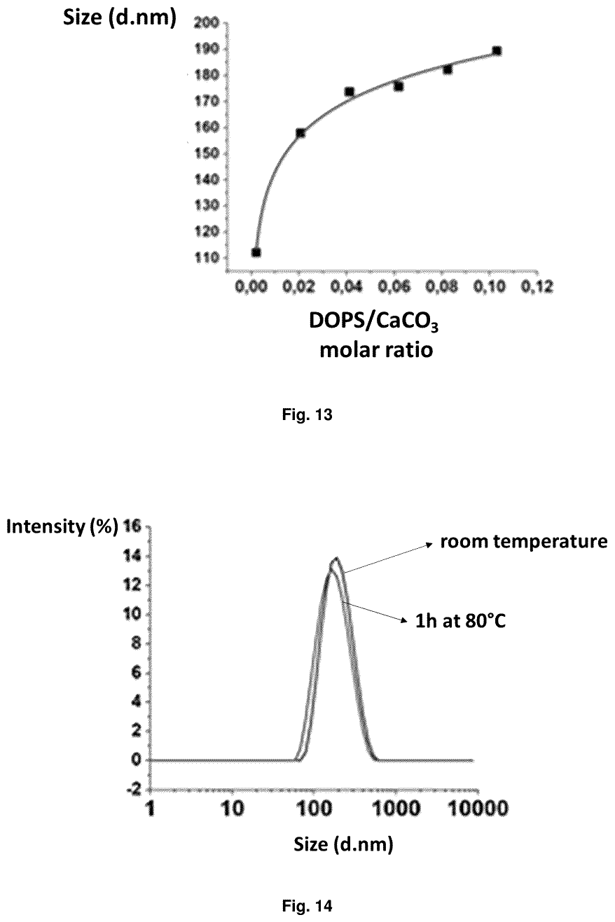

FIG. 13 shows the evolution of the size of the lipid-coated calcium carbonate nanoparticles in function of the weight ratio between DOPS and calcium carbonate of the calcium carbonate nanoparticles. Thus, the increase of DOPS concentration leads to a size increase and seems to follow a logarithmic fit.

FIG. 14 shows that the stability and the integrity of the amorphous calcium carbonate nanoparticles stabilized by DOPS at room temperature (i.e. about 20° C.) in water and after exposure of 1 hour at 70° C. have not been impaired.

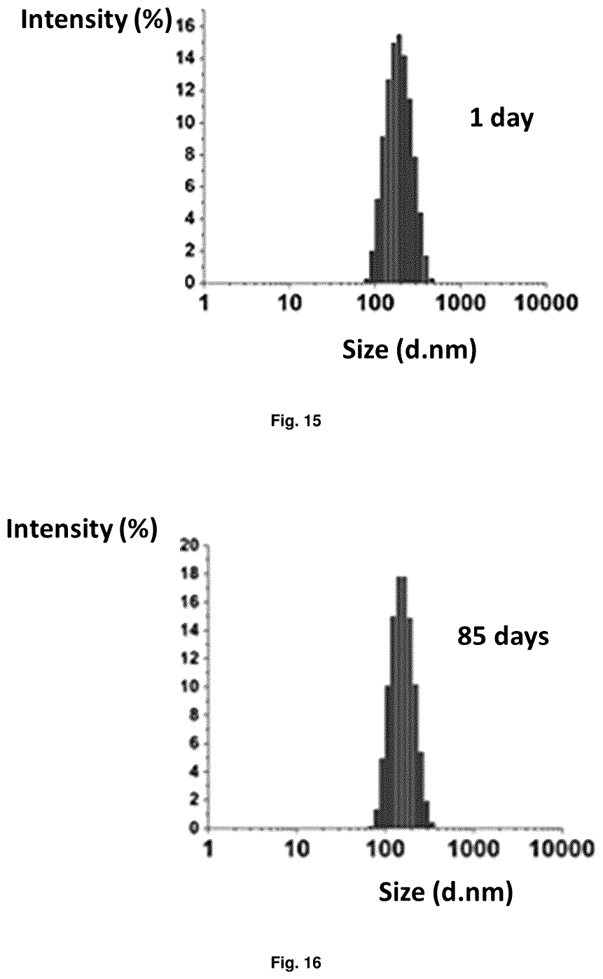

FIGS. 15 and 16 show the stability in water of the amorphous calcium carbonate nanoparticles stabilized by DOPS in term of the size dispersion respectively after 1 day and after 85 days. The dispersion remains narrow and no pH increase (indicative of the dissolution of the calcium carbonate) was observed.

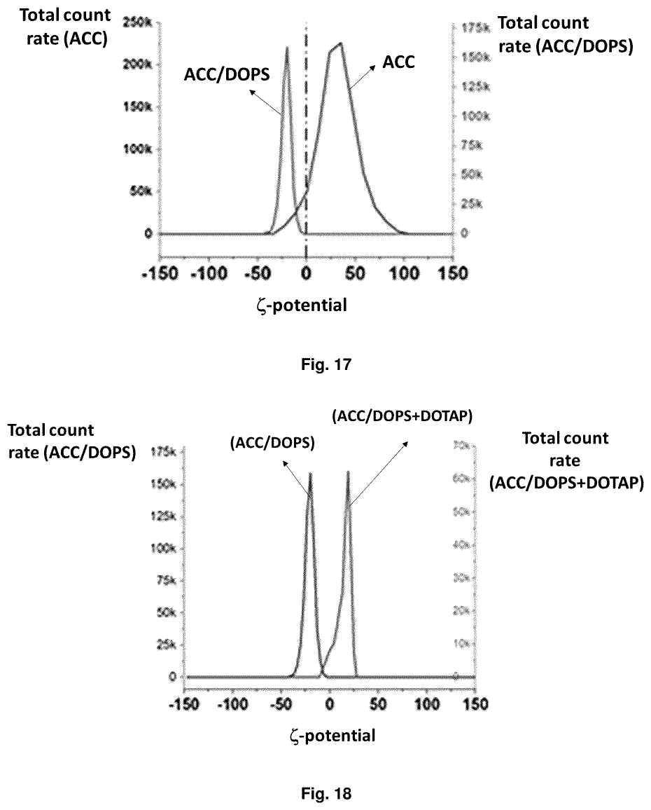

FIG. 17 shows that the positive electrical charge of the amorphous calcium carbonate nanoparticles in ethanol has, once the nanoparticles have been stabilized with DOPS, switched to a negative electrical charge. This switched testifies that the lipids are indeed structured around the calcium carbonate core of the nanoparticles. The presence of negatively charged non-PEGylated lipid multilayers around the calcium carbonate core of the nanoparticles enhances the versatility of the stabilized nanoparticles, since, as the lipids structuring the protective layer around the core of amorphous calcium carbonate are not sterically hindered, it is possible to further functionalize the amorphous calcium carbonate nanoparticles stabilized by DOPS and/or consider templating strategies in aqueous media using these objects as a template.

### Modulating of the Electrical Charge on the Lipid-Coated Amorphous Calcium Carbonate Nanoparticles

An ethanolic solution of DOTAP, at a concentration of 0.25 g/L has been prepared by dissolving DOTAP using ultrasonication in an ultrasonic bath working at 37 kHz of intensity and 100% power. Then this was injected with an automatic pipette into an aqueous solution of DOPS-coated amorphous calcium carbonate nanoparticles. The concentration of lipid-coated calcium carbonate nanoparticle is of 0.6 g/L. To achieve the stirring, several aspiration cycles with the automatic pipette are performed, followed by 15 seconds of a vortex. The resulting aqueous solution of (DOTAP+DOPS)-coated amorphous calcium carbonate nanoparticles is then stored in the fridge at 4° C.

FIG. 18 shows the ζ-potential shift, from negative values to positive values, proving, therefore, the insertion of the additional amphiphilic compound.

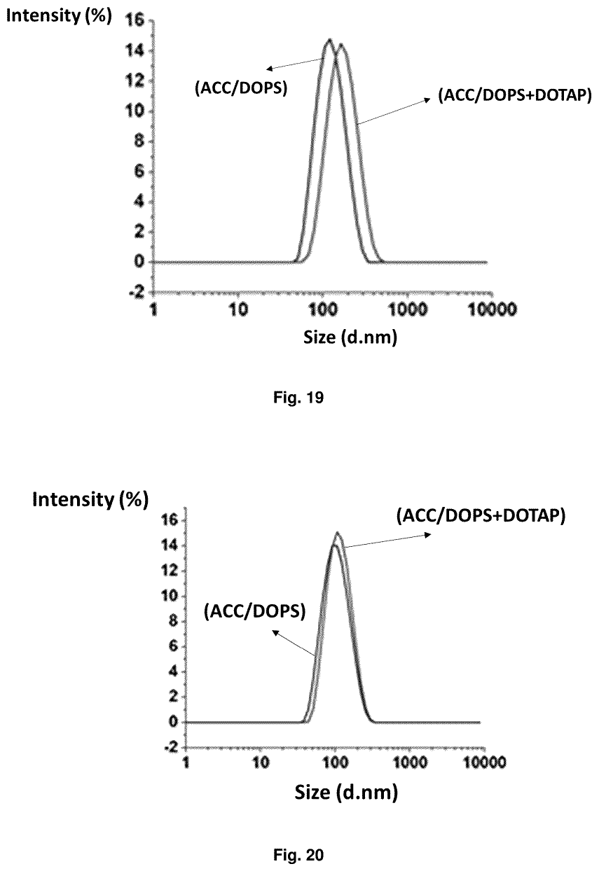

FIGS. 19 and 20 show the shift in the size distribution of the amorphous calcium carbonate nanoparticles stabilized by DOPS before and after post-functionalization with DOTAP, with respectively a DOPS/DOTAP weight ratio of 0.1 and 1. The size of the shift is larger when the DOTAP concentration is increased, revealing that the DOTAP quantity injected influences the final size of the nanoparticles. This observation proves that the additional amphiphilic compound, DOTAP, is structured around the core of the amorphous calcium carbonate in the lipid layers of DOPS.

1-28. (canceled)

29. Lipid-coated calcium carbonate nanoparticles, said nanoparticles comprising an outer layer and a core being calcium carbonate nanoparticle, wherein said core is one or more selected from vaterite, proto-vaterite, and amorphous calcium carbonate as determined by X-Ray diffraction, wherein said core is at least partially coated with one or more amphiphilic compounds wherein each amphiphilic compound has one hydrophilic head and at least one hydrophobic tail, characterized in that the hydrophilic heads of said one or more amphiphilic compounds are negatively charged and form the outer layer of the nanoparticles, in that the nanoparticles have a surface charge having a □-potential below 0 mV as determined by micro-electrophoretic light scattering technology, in that said one or more amphiphilic compounds are PEG-free, and in that said one or more amphiphilic compounds are lipids selected from lipids with a negatively charged head and with at least one hydrophobic tail.

30. Lipid-coated calcium carbonate nanoparticles according to claim 29, characterized in that said one or more amphiphilic compounds are lipids selected from phosphatidyl serine, phosphatidyl glycerol, phosphatidyl inositol and any mixture thereof.

31. Lipid-coated calcium carbonate nanoparticles according to claim 29, characterized in that at least a part of said one or more amphiphilic compounds is arranged into a plurality of bilayers with the negatively charged heads; said plurality of bilayers with the negatively charged heads being interconnected between each other.

32. Lipid-coated calcium carbonate nanoparticles according to claim 31, characterized in that said plurality of bilayers is infiltrated into the core of the calcium carbonate nanoparticles, the extent of the infiltration ranging between 5% and 40% of the core diameter as determined by CryoTEM analysis.

33. Lipid-coated calcium carbonate nanoparticles according to claim 29, characterized in that the outer layer formed by the hydrophilic heads of said one or more amphiphilic compounds has a thickness ranging between 5 nm and 100 nm as determined by CryoTEM analysis.

34. Lipid-coated calcium carbonate nanoparticles according to claim 29, characterized in that, when dispersed in water or aqueous media, said nanoparticles are monodisperse.

35. Lipid-coated calcium carbonate nanoparticles according to claim 29, characterized in that, when dispersed in water or aqueous media, said nanoparticles have a polydispersity index inferior to 0.20 as determined by dynamic light scattering.

36. Lipid-coated calcium carbonate nanoparticles according to claim 29, characterized in that said nanoparticles have an average diameter size ranging between 50 nm and 150 nm as determined by scanning electron microscopy.

37. Lipid-coated calcium carbonate nanoparticles according to claim 29, characterized in that said one or more amphiphilic compounds are selected among 2-dioleoyl-sn-glycero-3-phospho-L-serine; 1,2-dihexadecanoyl-sn-glycero-3-phospho-L-serine; 1,2-dipalmitoyl-sn-glycero-3-phospho-rac-(1-glycerol); 1,2-diacyl-sn-glycero-3-phospho-1-rac-gylcerol; 1,2-dioleoyl-sn-glycero-3-phospho-1′-myo-inositol; 1,2-dipalmitoyl-sn-glycero-3-phospho-1′-myo-inositol; and any mixture thereof.

38. Lipid-coated calcium carbonate nanoparticles according to claim 29, characterized in that the nanoparticles further comprise one or more additional amphiphilic compounds wherein each additional amphiphilic compound has one hydrophilic head and at least one hydrophobic tail, wherein said hydrophilic heads of said one or more additional amphiphilic compounds are positively charged; wherein the external surface of the outer layer of the nanoparticles comprises positively charged heads of the one or more additional amphiphilic compounds in addition to the negatively charged heads of the one or more amphiphilic compounds, wherein said positively charged heads being inserted between the negatively charged heads.

39. Lipid-coated calcium carbonate nanoparticles according to claim 29, characterized in that the nanoparticles further comprise one or more additional amphiphilic compounds wherein each additional amphiphilic compound has one hydrophilic head and at least one hydrophobic tail, wherein said hydrophilic heads of said one or more additional amphiphilic compounds are positively charged, wherein at least a part of said one or more additional amphiphilic compounds is arranged into one or more bilayers with positively charged heads, said one or more bilayers with the positively charged heads being interconnected between each other and covering at least partially the outer layer of said nanoparticles.

40. Lipid-coated calcium carbonate nanoparticles according to claim 39, characterized in that said one or more bilayers with the positively charged heads have a thickness comprised between 10 nm and 50 nm as determined by dynamic light scattering.

41. Lipid-coated calcium carbonate nanoparticles according to claim 38, characterized in that said one or more additional amphiphilic compounds are selected from 1,2-dioleoyl-3-trimethylammonium-propane and/or 1,2-dioleoyl-sn-glycero-3-ethylphosphocholine.

42. Lipid-coated calcium carbonate nanoparticles according to claim 38, characterized in that the outer layer of the nanoparticles further comprising one or more additional amphiphilic compounds has a ζ-potential ranging between −25 mV up to +50 mV as determined by micro-electrophoretic light scattering technology.

43. Method for forming lipid-coated calcium carbonate nanoparticles, said method being characterized in that it comprises the following steps:
a) providing calcium carbonate nanoparticles, to form a core selected from one or more of vaterite, proto-vaterite, and amorphous calcium carbonate as determined by X-Ray diffraction;
b) dissolving at least one amphiphilic compound in ethanol, to form an ethanolic solution of at least one amphiphilic compound, wherein said one or more amphiphilic compounds are PEG-free and wherein said one or more amphiphilic compounds are lipids selected from lipids with a negatively charged head and with at least one hydrophobic tail;
c) mixing said calcium carbonate nanoparticles provided in step (a) with said ethanolic solution of at least one amphiphilic compound formed in step (b) to form a mixture;
d) injecting said mixture in water to provide a solution of lipid-coated calcium carbonate nanoparticles
wherein said nanoparticles comprise an outer layer and a core being calcium carbonate nanoparticle, wherein said core is one or more selected from vaterite, proto-vaterite, and amorphous calcium carbonate as determined by X-Ray diffraction, wherein said core is at least partially coated with one or more amphiphilic compounds wherein each amphiphilic compound has one hydrophilic head and at least one hydrophobic tail, wherein the hydrophilic heads of said one or more amphiphilic compounds are negatively charged and form the outer layer of the nanoparticles, wherein the nanoparticles have a surface charge having a ζ-potential below 0 mV as determined by micro-electrophoretic light scattering technology, wherein said one or more amphiphilic compounds are PEG-free, and wherein said one or more amphiphilic compounds are lipids selected from lipids with a negatively charged head and with at least one hydrophobic tail.

44. Method according to claim 43, characterized in that it further comprises the step (e) of recovering said lipid-coated calcium carbonate nanoparticles.

45. The method according to claim 43, characterized in that the weight ratio between the at least one amphiphilic compound and calcium carbonate of the calcium carbonate nanoparticles is ranging between 0.01 and 1.

46. Method for modulating the electrical charge of lipid-coated calcium carbonate nanoparticles, characterized in that said method for modulating the electrical charge comprises the following steps:
a) providing the lipid-coated calcium carbonate nanoparticles wherein said nanoparticles comprise an outer layer and a core being calcium carbonate nanoparticle, wherein said core is one or more selected from vaterite, proto-vaterite, and amorphous calcium carbonate as determined by X-Ray diffraction, wherein said core is at least partially coated with one or more amphiphilic compounds wherein each amphiphilic compound has one hydrophilic head and at least one hydrophobic tail, wherein the hydrophilic heads of said one or more amphiphilic compounds are negatively charged and form the outer layer of the nanoparticles, wherein the nanoparticles have a surface charge having a ζ-potential below 0 mV as determined by micro-electrophoretic light scattering technology, wherein said one or more amphiphilic compounds are PEG-free, and wherein said one or more amphiphilic compounds are lipids selected from lipids with a negatively charged head and with at least one hydrophobic tail;
b) dissolving said lipid-coated calcium carbonate nanoparticles provided in step (a) in water to form an aqueous solution of lipid-coated calcium carbonate nanoparticles;
c) dispersing at least one additional amphiphilic compound having one hydrophilic head and at least one hydrophobic tail in ethanol to form an ethanolic solution of said at least one additional amphiphilic compound; wherein said hydrophilic head of said at least one additional amphiphilic compound is positively charged;
d) injecting the ethanolic solution of said at least one additional amphiphilic compound of step (c) into the aqueous solution of lipid-coated calcium carbonate nanoparticles of step (b) to form lipid-coated calcium carbonate nanoparticles
wherein the lipid-coated calcium carbonate nanoparticles further comprise one or more additional amphiphilic compounds wherein each additional amphiphilic compound has one hydrophilic head and at least one hydrophobic tail, wherein said hydrophilic heads of said one or more additional amphiphilic compounds are positively charged; wherein the external surface of the outer layer of the nanoparticles comprises positively charged heads of the one or more additional amphiphilic compounds in addition to the negatively charged heads of the one or more amphiphilic compounds, wherein said positively charged heads being inserted between the negatively charged heads.

47. The method according to claim 46, characterized in that it further comprises a step (e) of recovering said lipid-coated calcium carbonate nanoparticles.

48. The method according to claim 46, characterized in that the weight ratio between the at least one amphiphilic compound and the at least one additional amphiphilic compound is ranging between 0.05 and 2.
